(19) United States
(12) Patent Application Publication
(10) Pub. No.: US 2005/0089864 A1
(43) Pub. Date: Apr. 28, 2005

(54) Signalling aptamer complexes

(76) Inventors: Yingfu Li, Dundas (CA); Razan Nutiu, Hamilton (CA)

Correspondence Address: JAGTIANI + GUTTAG, 10363-A DEMOCRACY LANE, FAIRFAX, VA, 22030, US

(21) Appl. No.: 10/502,190

(22) PCT Filed: Jan. 22, 2003
(86) PCT No.: PCT/CA03/00086

(30) Foreign Application Priority Data
Jan. 22, 2002 (US) 60349340

(51) Int. Cl. C12Q 1/68; C07H 21/04

(52) U.S. Cl. 435/6; 536/24.3; 536/25.32

(57) ABSTRACT

Aptamer based fluorescent reporters that function based on a switch from DNA/DNA duplex conformation to DNA/target conformation are provided. The DNA/DNA duplex is formed between the aptamer DNA sequence and an oligonucleotide carrying a reporter moiety. When the aptamer target is present, the aptamer assumes a tertiary structure for binding to the target. The formation of the tertiary structure forces the dissociation of the duplex structure and a signal is generated. The signal is preferably a fluorescent signal due to spatial separation of a fluorophore/quencher pair.

### FIELD OF THE INVENTION

The present invention is directed to signalling aptamer complexes and methods of making the same.

### BACKGROUND OF THE INVENTION

Throughout this application, various references are cited in parentheses to describe more fully the state of the art to which this invention pertains. The disclosures of these references are hereby incorporated by reference into the present disclosure, and for convenience the references are listed in the appended list of references. Aptamers are single-stranded nucleic acids that are isolated from random-sequence DNA or RNA libraries by in vitro selection (Tuerk & Gold, 1990; Ellington & Szostak, 1990). A large number of DNA or RNA sequences have been isolated which bind a diverse range of targets, including small molecules (metal ions and simple organic compounds), biological cofactors (nucleotides, amino acids, and peptides), macromolecules (proteins and nucleic acids), and even entire organisms. Aptamers can be in the form of single stranded DNA, RNA, or modified nucleic acids. They typically contain 15 to 60 nucleotides and can be inexpensively synthesized.

It has been well documented that aptamers can be made to have very high affinity. For example, a 24-nt RNA aptamer carrying several 2-aminopyrimidine modifications was selected for binding to vascular permeability factor/vascular endothelial growth factor (VPF/VEGF) with an observed Kd of 0.14 nM (Green et al., 1995). Similarly, DNA aptamers have been isolated which bind to platelet-derived growth factor (PDGF)-AB with subnanomolar affinity (Green et al., 1996). Recently, a series of 2′-fluoro modified RNA molecules were isolated that bind the human keratinocyte growth factor with Kd of approximately 0.3-3 pM (Pagratis et al., 1997).

Aptamers can also exhibit high specificity. An RNA aptamer isolated for theophyllin recognition shows more than 10,000-fold discrimination against caffeine, which differs from theophyllin by a single methyl group (Jenison et al., 1994). An RNA aptamer selected for binding to L-arginine has a 12,000 fold reduction in affinity to the D-arginine (Geiger et al., 1996). The target versatility and the high binding affinity of both DNA and RNA aptamers, their abilities in precision molecular recognition, along with the simplicity of in vitro selection methods, make DNA and RNA aptamers attractive bioanalytical and diagnostic tools. In particular, aptamer based biosensors and bioanalytic assays to distinguish specific analyte binding without the need for separation of aptamer-target complex have great potential in clinical and biomedical applications where rapid and simple analysis techniques are required desired. To this end, aptamers that signal by fluorescence are highly desirable. Since DNA and RNA do not contain any fluorescent group, standard aptamers lack intrinsic fluorescence signaling ability and have to be modified with external fluorophores. Three different approaches have been reported for generating fluorescence signaling aptamers. The first method was to modify aptamers with a single fluorophore to create aptamers that perform fluorescence signaling by conformational change between unbound and bound states. In an early effort, two different anti-adenosine aptamers, one made of RNA and one of DNA, were modified with acridine and tested for fluorescence enhancement (Jhaveri et al., 2000a). Although the approach was successful, only a small increase in fluorescence intensity (ca. 25-40%) was observed with saturating (10 mM) ATP. In a later attempt, Jhaveri et al. took a direct selection approach to isolate fluorescent signaling aptamers for ATP binding from an RNA pool that contained lowly incorporated fluoresceinated uridines (Jhaveri et al., 2000b). Although several aptamers failed to register fluorescence enhancement, one aptamer showed 100% fluorescence intensity increase at saturating concentrations of ATP.

The second approach involves the labeling of aptamers with fluorophores, followed by fluorescence-anisotropy measurements of the aptamer-target. A detection method, which uses glass surface-attached aptamers to specifically bind thrombin, has been described (Potyrailo et al., 1998). The thrombin-binding DNA aptamer was specially labeled with fluorescein and immobilized on a glass surface. The thrombin binding is detected by anisotrophic changes in fluorescence. Although this approach has several significant advantages (Hesselberth et al., 2000), it also comes with some drawbacks, including low sensitivity, incompatibility for small molecule detection, time consuming, and inability for parallel detection.

The third methodology is directed at formulating aptamers into molecular beacons. A molecular beacon is an oligonucleotide doubly modified with a fluorophore and a quencher at its two termini. The fluorophore (F) can emit intensive fluorescence when it is excited, and the quencher (Q) is nonfluorescent but can engage in fluorescence resonance energy transfer (FRET) with the fluorophore to quench its fluorescence. A molecular beacon adopts a closed-state, stem-loop structure where the fluorophore and the quencher are situated in close proximity, resulting in fluorescence quenching. In the presence of a nucleic acid target that contains the sequence complementary to the loop, the molecular beacon adopts an open state structure where the fluorophore and quencher are separated, leading to the restoration of fluorescence (Tyagi and Kramer, 1996). It has been shown that aptamers can be converted into aptamer beacons modified with a fluorophore-quencher pair. In the absence of the target, the aptamer beacon forms the stem-loop structure to engage the fluorophore and the quencher in fluorescence quenching. In the presence of the target, the aptamer-target complex formation induces a structure transition that causes the separation of the fluorophore and the quencher, leading to the regeneration of fluorescence. An anti-thrombin aptamer has been engineered to obtain the aptamer beacon by adding nucleotides to the 5′-end which are complementary to nucleotides at the 3′-end of the aptamer (Hamaguchi et al., 2001). In the absence of thrombin, the added nucleotides form a duplex with the 3′-end, forcing the aptamer beacon into a stem-loop structure with minimal fluorescence signal. In the presence of thrombin, the aptamer beacon forms the ligand-binding structure with the fluorophore and quencher located far apart, resulting in significant fluorescence enhancement. Yamamoto et al. adopted a different aptamer beacon approach to analyze the Tat protein of HIV (Yamamoto et al., 2000). They split a Tat-binding RNA aptamer into two RNA molecules, one of which was converted into a molecular beacon where the fluorophore and quencher were attached onto the 5′- and 3′-ends of the RNA that forms a hairpin structure. In the absence of Tat, the two RNA molecules exist independently and the molecular beacon half of the aptamer adopts stem-loop structure, resulting in fluorescence quenching. When Tat is introduced into the solution, the two RNA oligomers engage in tertiary interaction with Tat, causing the separation of the fluorophore and the quencher, which leads to significant enhancement of fluorescence.

Although the above strategies are successful in creating signaling aptamers, there is still a great demand for a generally adaptable methodology to easily and cost-effectively convert any nonfluorescent aptamer into very sensitive fluorescent reporter. Not only will a universal and cost-effective converting system facilitate the use of individual signaling aptamers in diagnostic and bioanalytical applications, it will also allow the construction of aptamer arrays or multiplexing aptamer biosensors for a variety of high throughput applications including the profiling of proteins and metabolites from healthy and diseased cells.

### SUMMARY OF THE INVENTION

The present invention is directed to novel detection moieties based on an aptamer sequence. Specifically, signalling aptamer complexes are provided which comprise a first oligonucleotide having an aptamer sequence with a target-binding domain and at least one additional oligonucleotide capable of forming a duplex structure with a portion of said first oligonucleotide, wherein a reporter signal is emitted when the duplex structure is dissociated when the target-binding domain of the aptamer interacts with a target molecule. Reporter molecules include, but are not limited to, fluorescent and/or quencher reporters, radioactive reporters, luminescent reporters, chromogenic reporters, and density reporters such as gold particles. The signalling aptamer complex can be provided in a pre-assembled (i.e. duplex) format or the components can be added together in a detection assay.

In one aspect of the invention, there is provided a signalling aptamer complex for the detection of a target, the aptamer complex comprising:
i) a first oligonucleotide having a target binding domain, and
ii) at least one additional oligonucleotide having a sequence
complementary to a region of the first oligonucleotide, wherein in the absence of the target, complementary regions of the first oligonucleotide and the additional oligonucleotide form a duplex structure and wherein in the presence of the target, the duplex structure dissociates and a reporter signal is generated.

In one embodiment, the first oligonucleotide is labeled with a fluorophore and the additional oligonucleotide has a quencher moiety associated therewith.

In another embodiment, the first oligonucleotide has a quencher moiety and the additional oligonucleotide is labeled with a fluorophore.

In a preferred embodiment, the first oligonucleotide comprises an FDNA binding domain capable of forming a duplex with a fluorophore modified oligonucleotide (FDNA).

In another preferred embodiment, the first oligonucleotide comprises 3-10 nucleotides inserted adjacent to the target binding domain wherein the nucleotides participate in the duplex formed between the first oligonucleotide and the additional oligonucleotide.

In a further aspect of the invention, the first oligonucleotide comprises an ATP-binding domain or a thrombin-binding domain.

In yet another aspect of the invention, there is provided a signalling aptamer complex for detection of a target, the aptamer complex comprising:
i) a first oligonucleotide having a target binding domain and a tagging domain,
ii) a second oligonucleotide labeled with a fluorophore and having a sequence complementary to the tagging domain, and
iii) a third oligonucleotide modified with a quencher and having a sequence complementary to a region of the target binding domain,
wherein in the absence of a target, a first duplex is formed between the second oligonucleotide and the tagging domain and a second duplex is formed between the third oligonucleotide and a segment of the target binding domain whereby the quencher and the fluorophore are sufficiently close to one another to quench a fluorescent signal.

In a preferred embodiment, the first oligonucleotide includes additional nucleotides intermediate the target binding domain and the tagging domain and the third oligonucleotide is complementary to and forms the second duplex with the additional nucleotides and the adjacent portion of the target binding domain.

In the presence of a target, the first oligonucleotide assumes a tertiary structure and the third oligonucleotide dissociates from the first oligonucleotide and a fluorescent signal is detectable.

In another aspect, a second oligonucleotide modified with a quencher and having a sequence complementary to the tagging domain, and a third oligonucleotide labeled with a fluorophore and having a sequence complementary to a region of the target binding domain, wherein in the absence of a target, a first duplex is formed between the second oligonucleotide and the tagging domain and a second duplex is formed between the third oligonucleotide and a segment of the target binding domain whereby the quencher and the fluorophore are sufficiently close to one another to quench a fluorescent signal.

In yet another aspect, there is provided a signalling aptamer complex comprising:
i) a first oligonucleotide having a target binding domain
ii) a second fluorphore-labeled oligonucleotide hybridized to a first segment of the target binding domain, and
iii) a third quencher-modified oligonucleotide hybridized to a second segment of the target binding domain adjacent to the first segment.

In a preferred embodiment, the flurophore labeled oligonucleotide comprises two fluorophores capable of exhibiting fluorescence energy transfer.

In another aspect, a method for modifying an aptamer into a signalling aptamer is provided. The method comprises interacting a reporter oligonucleotide, having a nucleotide sequence complementary to a target binding segment of the aptamer, with the aptamer to form a duplex structure.

In a preferred embodiment, the aptamer is labeled with a fluorophore and the reporter oligonucleotide is modified with a quencher.

In another aspect, a method for detecting the presence of a target is provided. The method comprises providing a signalling aptamer complex, interacting the complex with a target solution; and measuring a signal.

In a further aspect, there is provided a modified aptamer comprising a target binding domain and an oligonucleotide binding domain fused at one end. In yet another aspect of the invention, there is provided a signalling aptamer comprising an aptamer sequence and an oligonucleotide binding domain sequence fused at one end of the aptamer sequence. The oligonucleotide binding domain is also referred to as a tagging domain since it is used to tag on an additional oligonuleotide to the complex. Preferably the binding domain sequence is complementary to the sequence of a second oligonucleotide having a reporter molecule attached thereto.

In a particularly preferred embodiment, the present invention provides a generally applicable method that can be used to provide any DNA or RNA aptamer with a fluorescence signalling capability. The method involves the use of three oligomers: a) a modified aptamer denoted MAP, b) a fluorophore containing oligonucleotide termed FDNA, and c) a quencher modified oligonucleotide termed QDNA. Aptamers include a sequence capable of binding to a target or ligand. The FDNA and QDNA form duplexes with complementary regions of the modified aptamer.

Throughout this application, the terms oligonucleotide binding domain, tagging domain and FB domain are used interchangeably to refer to a sequence on a modified aptamer that is capable of forming a duplex with a second or fluorophore labeled oilgoncucleotide. QDNA is specially designed to form a weak duplex with the MAP. In the absence of the target, both FDNA and QDNA bind MAP and position the fluorophore and the quencher in close physical proximity, resulting in the fluorescence quenching. When the target of the aptamer is introduced into the solution, the binding domain of MAP rejects QDNA in favour of the formation of the tertiary structure for target binding. This gives rise to the fluorescence signalling by a de-quenching mechanism.

### BRIEF DESCRIPTION OF THE DRAWINGS

Preferred embodiments of the invention are described below with reference to the drawings, wherein:

FIG. 1 is a schematic diagram illustrating a signalling aptamer complex;

FIG. 2A illustrates the structure and FIG. 2B demonstrates the activity of a test aptamer complex;

FIG. 3A illustrates the composition of several signalling aptamer complexes;

FIG. 3B demonstrates the thermal denaturation profiles of the aptamer complexes shown in FIG. 3A;

FIG. 4A illustrates the oligonucleotides used to assemble ATP reporter A;

FIG. 4B illustrates the results of temperature-changing fluorescent assay in the presence or absence of ATP;

FIG. 4C is a tabular representation of the time to one-half maximal fluorescence in relation to temperature;

FIG. 5 is a bar graph illustrating the target specificity of the signalling aptamer complex;

FIG. 6A is a bar graph illustrating the effect of mutations on signalling capacity;

FIG. 6B illustrates the sequences of two mutant aptamers;

FIG. 7 illustrates the composition of three alternative signalling aptamer complexes;

FIG. 7A is a bar graph demonstrating the target specificity for four different signalling aptamer complexes;

FIG. 8A illustrates the oligonucleotides used in the construction of another signalling aptamer complex termed ATP Reporter E;

FIG. 8B is a graphical representation of the ATP Reporter E real-time detection at various temperatures;

FIG. 8C is a graphical representation of ATP Reporter E real-time detection as a function of ATP concentration;

FIG. 9A illustrates graphically the target detection range of ATP Reporter E;

FIG. 9B illustrates the target specificity of ATP Reporter E;

FIG. 10A illustrates the structure of a signalling anti-thrombin aptamer complex;

FIG. 10B illustrates the detection capability of the aptamer complex at various temperatures;

FIG. 10C illustrates the target detection range of the signalling anti-thrombin aptamer complex;

FIG. 10D illustrates the effect of Mg concentration on the time required to reach one-half maximal fluorescence;

FIG. 11 illustrates the signalling specificity of the signalling thrombin reporter;

FIG. 12 is a series of schematics illustrating modification schemes;

FIG. 13 illustrates a further series of modification schemes;

FIG. 14 illustrates a multiplexing assay;

FIG. 15 illustrates an exemplary array configuration;

FIG. 16 illustrates an optical sensor assay; and

FIG. 17 demonstrates the use of wave-length shifting aptamers.

### DETAILED DESCRIPTION

Aptamers are DNA or RNA molecules that are randomly selected based on their ability to bind other molecules. They can bind to nucleic acid molecules, proteins, small organic compounds, and even entire organisms.

Aptamers can bind target molecules with extraordinary affinity and specificity and are much easier and cost-effective to make than other recognition molecules, such as antibodies. Thus, there are many potential uses for aptamers in biotechnology and medicine.

Aptamers can be linear, single-stranded DNA or RNA molecules that are able to bind complementary nucleic acid sequences to form Watson-Crick duplex structures. Although single-stranded nucleic acids are commonly thought of as linear molecules, they can, in fact, take on complex, sequence dependent, three-dimensional shapes. Aptamers are specially created to have well-defined tertiary structures for specific recognition of targets of interest. Thus, aptamers have the inherent ability to engage in the formation of two totally different structural forms, either a nucleic acid duplex or a three-dimensional target complex.

The present invention exploits the dual structural properties of aptamers to provide novel, aptamer reporters which signal in the presence of a target molecule. These are referred to herein as signaling aptamer complexes (SAC) modified aptamer complexes or reporters. A series of methods for converting aptamers into reporters are also provided. In particular, method for modifying aptamers into fluorescent signalling aptamer complexes are described.

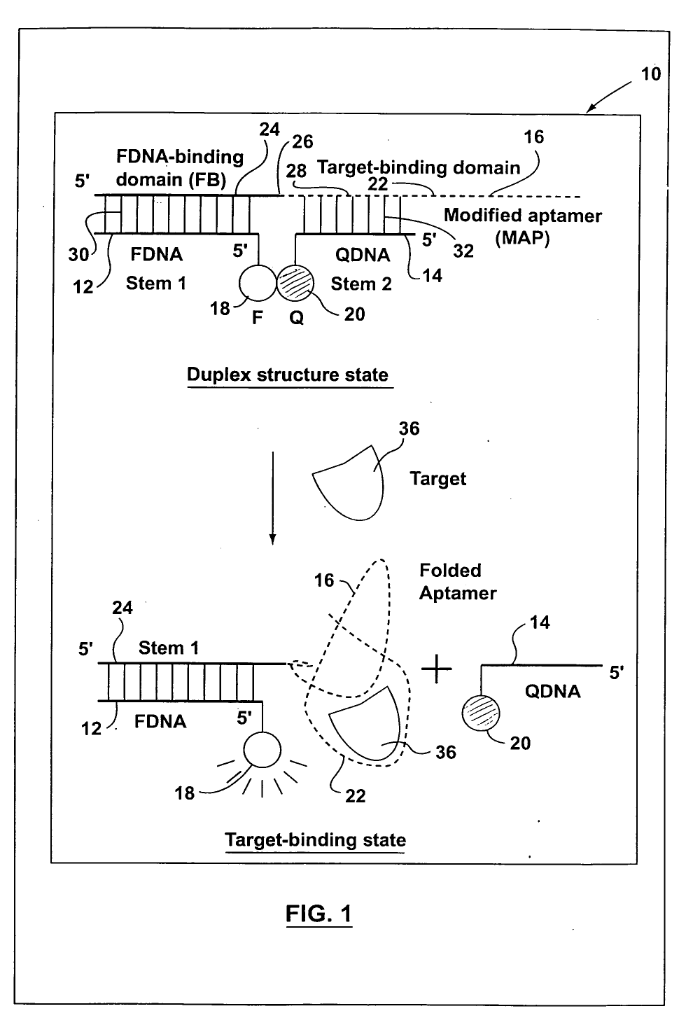

In one aspect of the invention, shown in FIG. 1, a signalling aptamer complex 10 comprises three oligomers: a) a fluorophore-containing oligonucleotide termed FDNA 12, b) a quencher modified oligonucleotide termed QDNA 14 and c) a modified aptamer denoted MAP 16. FDNA 12 is an oligonucleotide that contains a covalently linked fluorophore 18 at its 5′ end and can emit strong fluorescence when excited at certain wavelength. QDNA 14 contains a covalently attached quencher 20 at its 3′ end that engages in fluorescence resonance energy transfer (FRET) with the fluorophore 18 to quench its fluorescence. The modified aptamer oligonucleotide (MAP) 16, like a regular aptamer includes a target binding domain (TB domain) 22. The MAP further comprises an FDNA-binding domain (FB domain 24), fused onto the 5′ end 26 of the TB domain 22. The FB domain is also referred to as a tagging domain since it is used to tag on the flurophore labelled oligonucleotide. The QDNA oligonucleotide 14 has a sequence that is complementary to a segment of the MAP, termed the QB domain 28 and together they can form a duplex. The binding of FDNA 12 and QDNA 14 with MAP 16 results in the formation of stem 1 30 and stem 2 32, respectively. In this signalling aptamer complex, the fluorophore 18 and the quencher 20 are situated in close proximity and, as a result, the fluorophore does not emit fluorescence. In the presence of a target 36, on the other hand, the modified aptamer 16 adopts its tertiary structure conformation to bind the target. The formation of the tertiary structure forces the release of the QDNA from the signalling aptamer complex.

Consequently, the quencher is no longer located near the fluorophore and a fluorescent signal in emitted. Since the tagging FB domain 24 forms a rigid helical structure with FDNA 12, the FB domain does not affect the folding of the aptamer into its native tertiary structure nor does it significantly alter the binding capability of the target binding domain 22.

The relative strengths of stem 1 30 and stem 2 32 are important factors in the design of an effective signaling aptamer complex. Stem 1 (30) should be sufficiently robust that the FDNA 12 is strongly bound to the FB domain 24 of the MAP 16 to minimize the background fluorescent signal. One way to achieve a strong stem 1 is to incorporate a high GC content in the FDNA sequence.

Stem 2 (32), on the other hand, should have a strength less than that of stem 1 (30). The strength of stem 2 should be adequate to hold QDNA 14 onto the MAP 16 in the absence of target to provide a system with low background fluorescence due to the proximity of the quencher moiety and the fluorophore. It should not, however, be so strong that, in the presence of the target, the QDNA is not easily released to allow the formation of the tertiary structure required for target binding. In addition, a very high affinity between the QDNA and the QB domain could force the formed ligand-aptamer complex to dissociate, and lead to the preferential formation of the stem 2 duplex structure. If the interaction between the QDNA and the QB domain is too strong, the system either will not be able to produce strong fluorescence signal (due to quenching) or will not be able to hold steady fluorescence for an extended period of time needed for fluorescence measurement (due to competitive binding). A suitable QDNA for appropriate duplex formation can be established by screening QDNAs containing different numbers of base-pairs.

FIG. 1 represents only one embodiment of the invention and is used to illustrate the basic concept that structure switching from a duplex state to a tertiary conformation can be used to detect aptamer target binding. Although three oligonucloetides are shown in FIG. 1, it is clearly apparent that only two oligonucleotides are required to detect the switch in structure. These are the aptamer oligonucleotide and the oligonucleotide that participates in the duplex which is disrupted upon target binding. The duplexing oligonucleotide has a reporter moiety associated with it and is sometimes referred to as a reporter oligonucleotide. Signalling systems other than the fluorophore-quencher system can be used. The reporter moiety does not necessarily give an increase in signal. In some cases there may be a decrease in a signal when the reporter oligonucleotide dissociates from the aptamer sequence. In the example shown in FIG. 1, the quencher can be considered the reporter moiety since it is its movement that results in a change in signal.

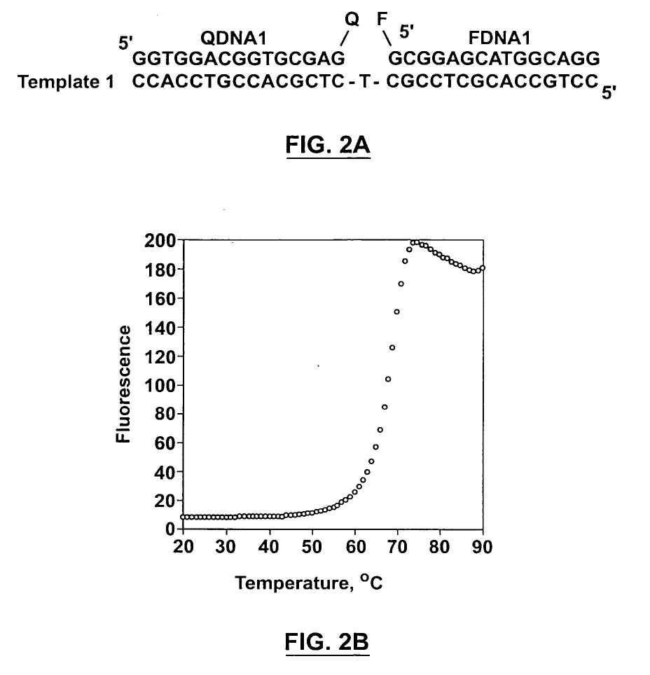

The feasibility of the system was demonstrated using various exemplary constructs. In one embodiment, illustrated in FIG. 2A and discussed further in Example 4, a test construct was prepared. The thermal denaturation profile of this construct is shown in FIG. 2B. This construct demonstrates that duplex formation does occur and that dissociation of at least one of the duplexes results in a fluorescent signal being generated.

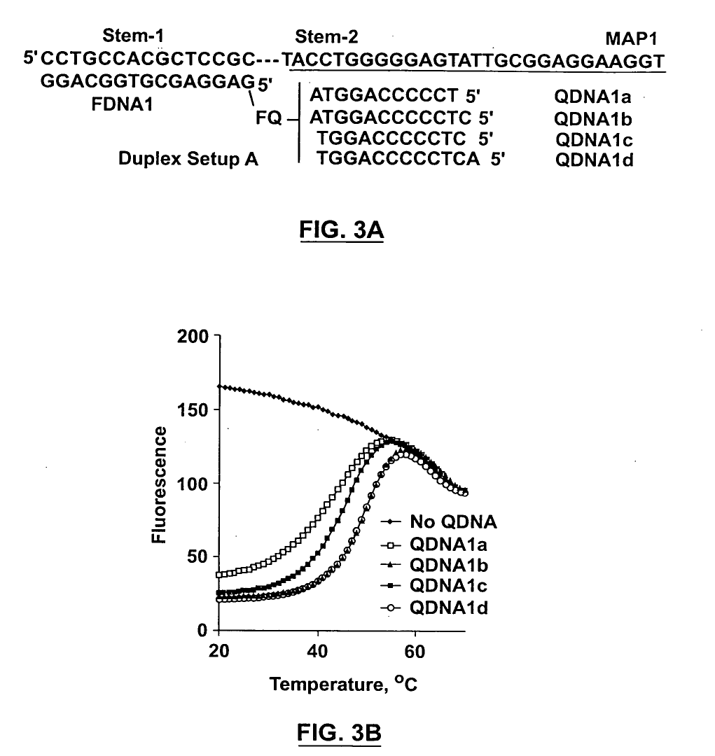

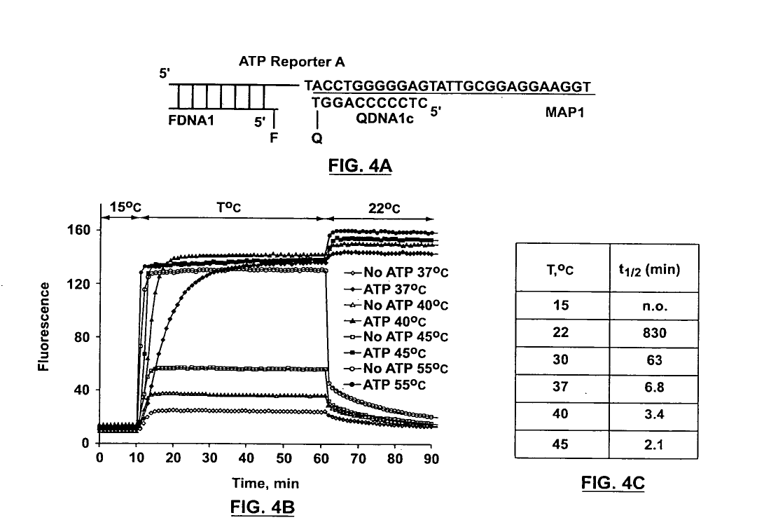

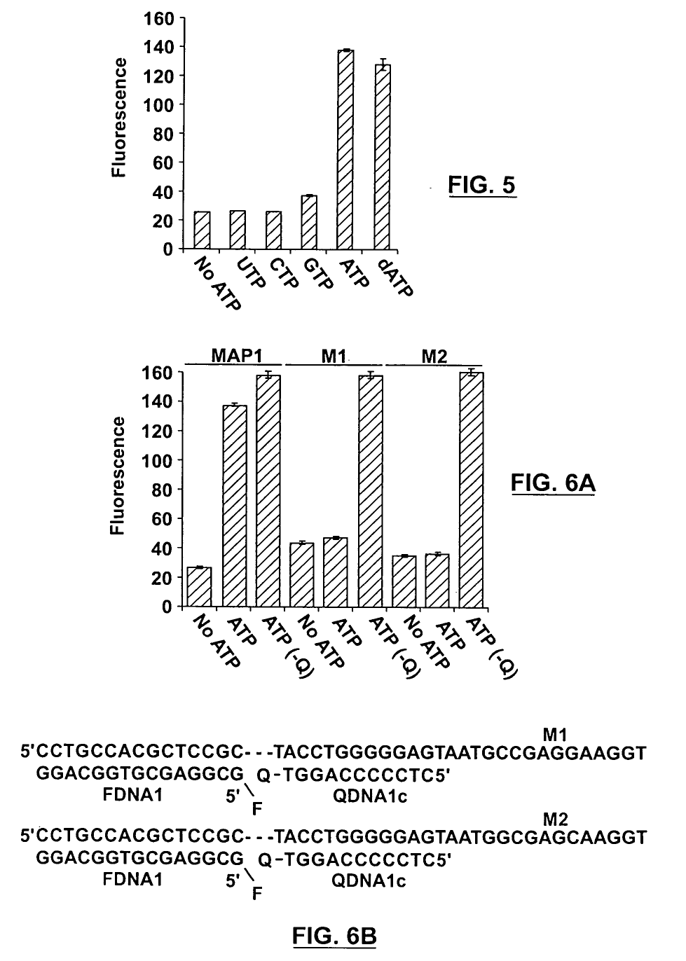

In another aspect of the invention, methods of preparing signalling aptamer complexes and the signalling complexes thus prepared are provided. In one preferred embodiment, a known aptamer oligonucleotide sequence is modified by fusing an FDNA binding domain at the 5′ end of the aptamer. A QDNA that has a sequence complementary to part of the target binding domain of the aptamer is synthesised. An appropriate QDNA sequence can be predicted based on the aptamer sequence and the thermal denaturation profiles of different QDNA sequences can be determined to select the most appropriate. An additional nucleotide is optionally inserted on the modified aptamer between the QDNA binding domain and the FDNA binding domain to address any potential steric hindrance problems that could affect binding of the aptamer to its target. When the aptamer sequence changes its structure to bind to a target, the QDNA duplex is disrupted and a fluorescent signal is generated. An exemplary signalling aptamer complex constructed in this manner and its properties are illustrated in FIGS. 3 to 6. The experimental details demonstrating the signalling properties of this aptamer are discussed in Examples 5 to 7. It is clearly apparent that while these examples refer to a modified ATP binding aptamer, any other aptamer can be modified in the same way to provide a signalling aptamer complex according to the present invention. The results indicate a signalling aptamer complex of this type has a good noise to signal ratio at temperatures appropriate for aptamer target binding (FIG. 4). In addition, the signal generated is target specific (FIG. 5) and no signal is generated when mutation which affect target binding are introduced in the aptamer sequence (FIG. 6).

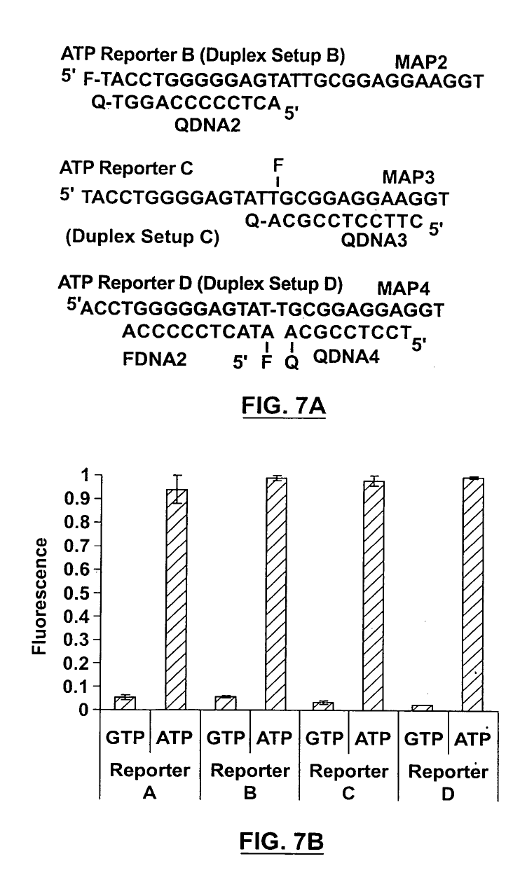

In another preferred embodiment, a signalling aptamer complex can be constructed by modifying the aptamer sequence to include a fluorophore at the 5′ end. In this type of construct, there is no need to provide an FDNA binding (FB) domain or an FDNA oligonucleotide since the “F” is directly linked to the aptamer sequence. A QDNA complementary to a region at the 5′ end of the aptamer sequence is provided. In the presence of its target the aptamer will undergo structure switching. When the aptamer assumes its tertiary conformation to interact with its target, the QDNA duplex will be disrupted and the quencher will be displaced away from the fluorophore. In this case the QDNA is the reporter oligonucleotide and the quencher is the reporter moiety since it is its effect that is being measured. An exemplary signalling aptamer complex designed in this way is shown in FIG. 7A under the name “ATP Reporter B” and discussed further in Example 8. The target specificity of this type of aptamer are shown in FIG. 7B.

In a further embodiment, a signalling aptamer complex is provided wherein an aptamer is modified with a fluorophore at an internal nucleotide. The modified aptamer forms a duplex with a QDNA having a sequence complementary to a region of the aptamer adjacent to the labeled nucleotide. An exemplary signalling aptamer of this type is shown in FIG. 7A under the title “ATP Reporter C”. The properties of this type of aptamer complex are shown in FIG. 7B and discussed in Example 8.

In yet another embodiment, a signalling aptamer complex is provided where the aptamer component is not modified. An FDNA is provided which has a sequence complementary to a segment of the native aptamer sequence and a QDNA is provided which has a sequence complementary to an adjacent segment of the aptamer sequence. When the FDNA and the QDNA form duplexes with the aptamer sequences, the QDNA is sufficiently close to the FDNA to quench the fluorescence. In the presence of target the QDNA, the FDNA or both are dissociated from the aptamer sequence and a fluorescent signal is generated. An example of this type of signalling complex is shown in FIG. 7A under the title “ATP Reporter D”. The signalling properties of this type of aptamer are shown in FIG. 7B and described in Example 8.

While many examples have been given where the dissociation of QDNA results in generation of a signal, it is clearly apparent that a signalling aptamer could be designed where dissociation of FDNA results in a signal. The only requirement for generation of a fluorescent signal is the spatial separation of the fluorophore and the quencher due to a change in the structure of the aptamer from a duplex state to a tertiary conformational state.

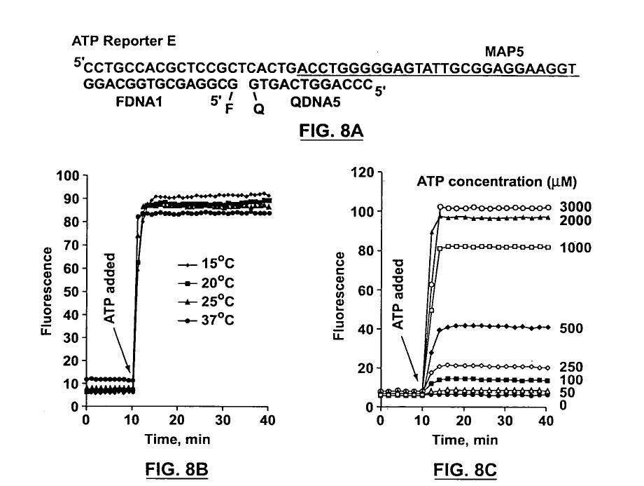

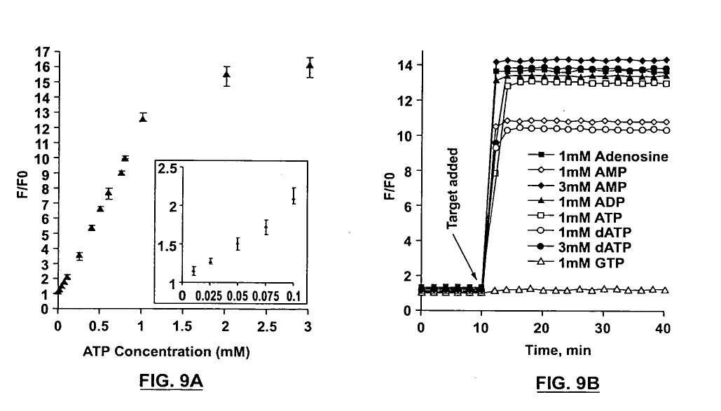

In another embodiment, a signalling aptamer complex is provided in which some additional nucleotides are inserted at one end of the aptamer sequence. Preferably 3 to 10 nucleotides are inserted. These additional nucleotides form part of the QDNA binding (QB) domain. A QDNA is provided which forms base pairs with the inserted nucleotides and a segment of the adjacent aptamer sequence. Addition of the extra nucleotides permits the use of a QDNA that has a good thermal denaturation profile and minimal effect on aptamer target binding. The modified aptamer may optionally include an FB domain or it may be labelled directly with a fluorophore. An exemplary aptamer of this type, named “ATP Reporter E” is shown in FIG. 8A and described further in Example 9. This aptamer has excellent real-time signalling capability (FIGS. 8B, 8C). The signal generated correlated well with the target concentration (FIG. 9A) and is target specific (FIG. 9B).

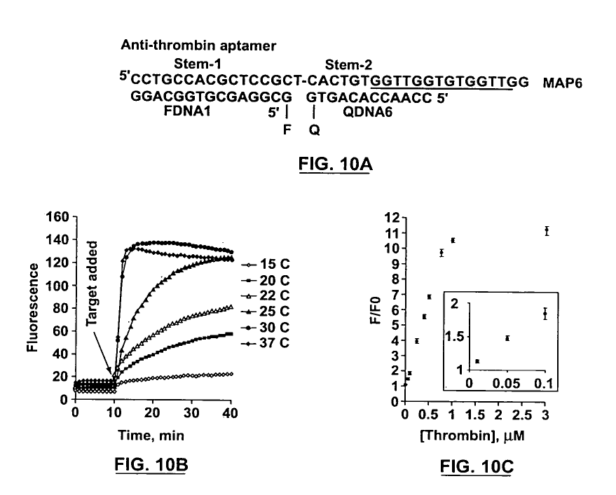

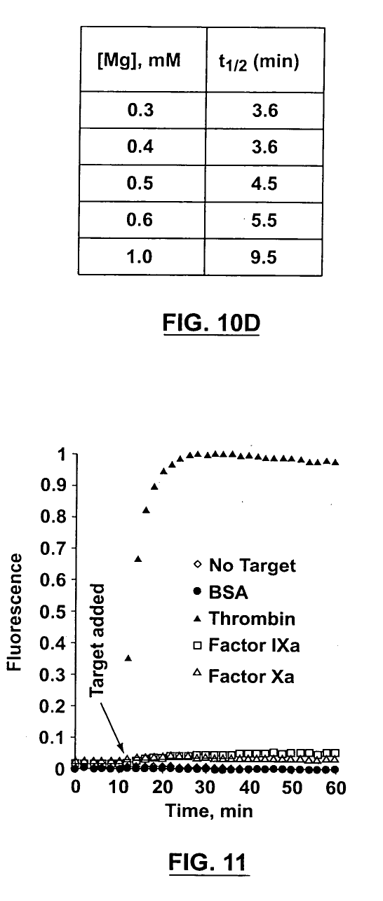

Another exemplary signalling aptamer having additional nucleotides inserted at one end of the aptamer sequence which form base pairs with a QDNA is shown in FIG. 10A. This aptamer is specific for thrombin and has excellent signalling properties as illustrated in FIGS. 10 and 11 and discussed further in Example 11.

Both the anti-ATP and anti-thrombin reporters exhibit a large signaling magnitude change. In addition, the signalling aptamer complexes retained the same target specificity as the original aptamers. The modification is applicable to both high affinity aptamers (e.g. the thrombin-binding aptamer) and low affinity aptamers (e.g. the ATP aptamer) as well as large and small sized aptamers. The successful engineering of several DNA aptamer reporters based on the same principle clearly demonstrates that the modification strategies can be easily adapted for the conversion of any DNA aptamer into a signalling aptamer complex.

The present invention takes advantage of the fact that an aptamer possesses two intrinsic structural properties: the ability to form a duplex structure with an externally supplied complementary single-stranded oligonucleotide and the ability to form a tertiary structure for ligand binding.

Since DNA and RNA aptamers all have the same dual structure capability, it is clearly apparent that the strategy used to generate the ATP-specific signalling apatmer complexes and the signalling thrombin aptamer is generally applicable for converting any nonsignaling aptamers into sensitive fluorescent reporters for detection of biological cofactors, metabolites, proteins and other ligands of interest. For example, an ATP-binding RNA aptamer or a thrombin-binding DNA aptamer can easily be converted into fluorescent reporters (i.e. signaling aptamer complex) using the same strategy described herein. The present invention thus encompasses any signalling aptamer complex prepared according to the methods described herein. It is clearly apparent that an aptamer can be modified in various ways to form a signalling aptamer complex in which a complementary oligonucleotide is dissociated from a duplex with the aptamer sequence when the aptamer assumes its tertiary structure in the presence of the target.

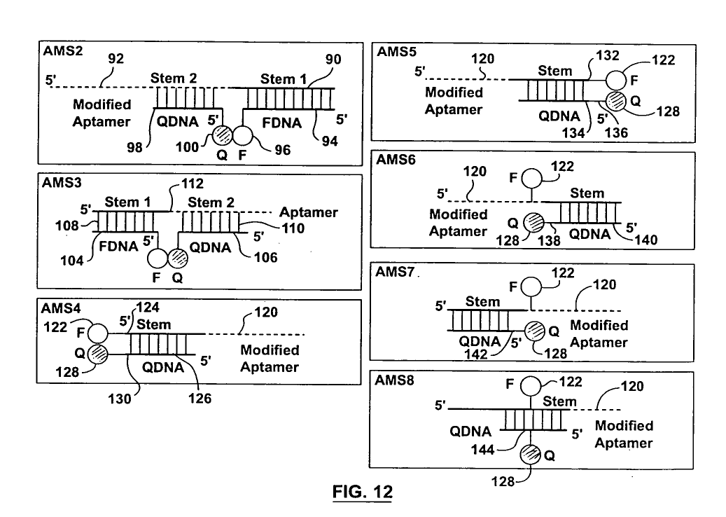

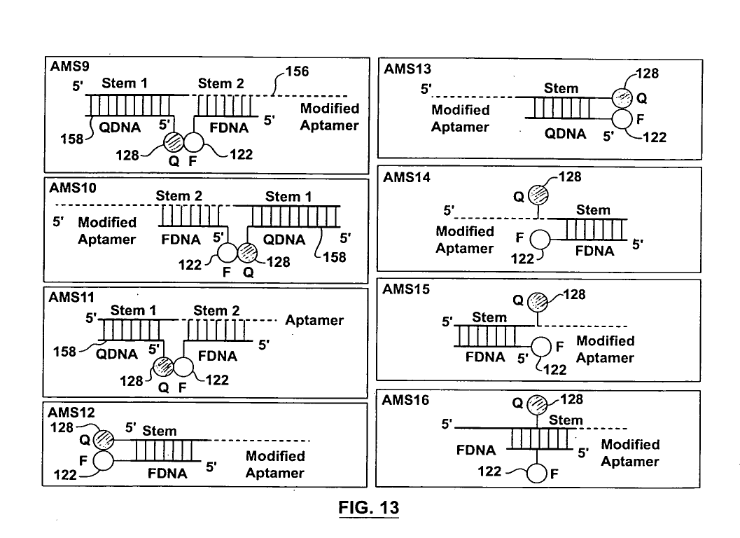

One skilled in the art would readily recognize that other signalling aptamer complex configurations could be designed where a switch in aptamer structure results in the generation of a signal. Modifications of the Aptamer Modification Scheme shown in FIG. 1 (AMS1) are encompassed within the scope of the present invention and several embodiments of the invention are shown in FIGS. 12 and 13. Signalling aptamer complexes created using any of the modification schemes are also included within the scope of the present invention.

Referring now to FIG. 12, Aptamer Modification Scheme 2 (AMS2) differs from AMS1 in the location of the FDNA-binding domain. In AMS1, the FDNA-binding domain is in front of the aptamer sequence, while in AMS2 the FDNA-binding domain 90 is located downstream of the aptamer sequence 92. In this configuration, the FDNA 94 has a 3′-fluorophore 96 and the QDNA 98 has a 5′-quencher 100. In AMS3, the FDNA 104 and QDNA 106 are specifically designed to form duplexes 108, 110 with an unmodified aptamer sequence 112. When the aptamer binds to the target, both the FDNA and the QDNA will be displaced. Since there is no complementarity between the FDNA and the QDNA, the fluorphore will become separated from the quencher and the solution will fluoresce.

Modification schemes, AMS4-8 all utilize an aptamer 120 that is covalently modified with the fluorophore 122. This eliminates the need for FDNA. In AMS4, the fluorophore 122 is attached onto the 5′-end 124 of the aptamer 120 and the QDNA 126 is modified with the quencher 128 at its 3′ end 130. In AMS5, the fluorophore 122 is attached at the 3′-end 132 of the aptamer 120 and the QDNA 134 has the quencher 128 attached at the 5′-end 136. The fluorophore 122 can also be attached onto a selected nucleotide within the aptamer sequence. In this conformation, the quencher 128 can be attached at the 3′-end 138 of the QDNA 140.(as in AMS6), the 5′-end 142 (as in AMS7) or at an internal nucleotide 144 (as in AMS8). AMS4-8 signal the target binding by rejecting the QDNA from the original duplex. It is clearly apparent that it is not essential that the fluorophore be covalently linked to the aptamer sequence and that, for all of the schemes presented herein, the oligonucleotides can be fluorescently labelled using other techniques and fluorophores other than fluorescein.

FIG. 13 illustrates eight more exemplary aptamer modification schemes. AMS9-16 are similar to AMS1-8 except that the positions of the fluorophore and the quencher are exchanged. In other words, in AMS1-8 the quencher 128 is always supplied via QDNA and the fluorophore 122 is either directly attached onto the aptamer or supplied indirectly through FDNA. In AMS9-16 the fluorophore 122 is always supplied through FDNA and the quencher 128 is either covalently linked with the aptamer 156 or noncovalently provided via QDNA 158. The fluorescence reporting for AMS9 10 and for AMS12-16 involves a structure transition mechanism that releases the FDNA from the initial duplex. Similar to AMS3, AMS11 signals the target binding by rejecting both the FDNA and the QDNA from the original duplex.

In another aspect of the invention, kits are provided for the modification of aptamers into signalling aptamer complexes. The kits are based on the modification schemes described throughout this description.

The signalling aptamer complexes of the present invention are useful molecular tools for the detection of biological cofactors, metabolites, proteins and a variety of other ligands. Real time detection can be performed using the signalling aptamer complexes of the present invention.

It is clearly apparent that the signalling aptamer complexes of the present invention can be provided as pre-assembled complexes (i.e. having a duplex structure) or the components can be added simultaneously to form a complex as they are being used. For example, QDNA and the target can be added simultaneously to a modified aptamer. Any free modified aptamer (i.e. not target bound) will associate with the QDNA.

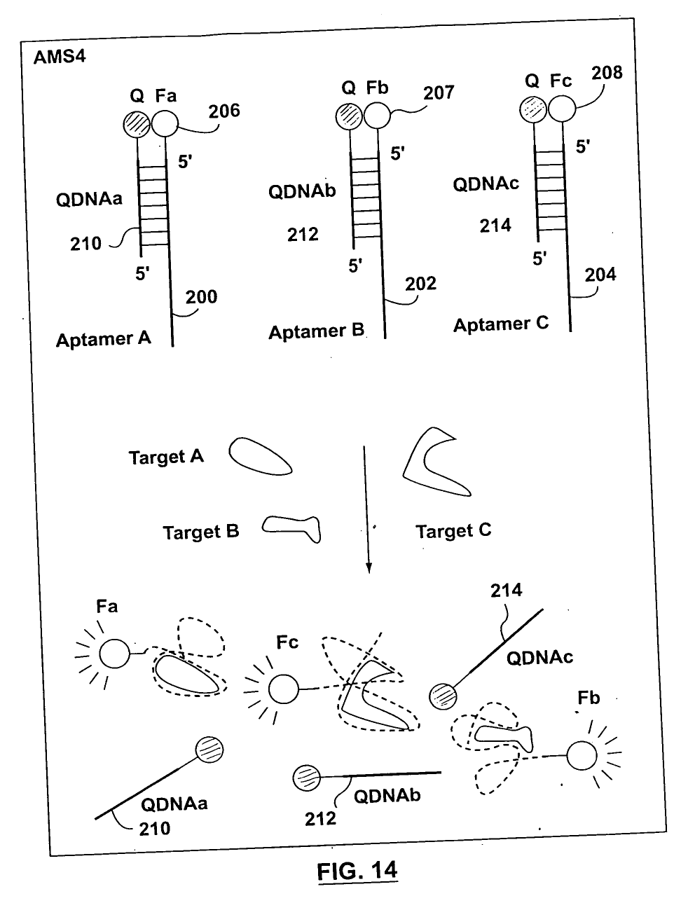

In another aspect of the invention, a multiplexing assay to detect different targets simultaneously is provided. Unlike other detection systems, the present system, which incorporates quencher/fluorophore pairs, does not require the separation of excess probes from target-aptamer complexes to obtain a good signal to noise ratio. FIG. 14 illustrates schematically an exemplary assay for the detection of three different targets using three signaling aptamer complexes prepared according to AMS4. Aptamers A 200, B 202, and C 204 are modified with three different fluorescent probes (Fa 206, Fb 207 or Fc 208, shown in blue, green and yellow, respectively) at the 5′-end. Each aptamer complex also includes a QDNA, QDNAa, 210 QDNAb 212 or QDNAc 214. In the presence of cognate targets, each signaling aptamer complex will undergo a target-induced structural transition. In the process, the QDNA 210, 212 or 214 is released and their respective fluorophore will emit fluorescence. Since the three aptamers are modified with three different fluorophores that emit fluorescence at different wavelength, individual targets in the solution can be identified easily by determining which fluorophore is dequenched. Although AMS4 is used as the example for multiplexing detection, it is clearly apparent that signaling aptamer complexes prepared according to any of the modification schemes described can also be used in multiplexing.

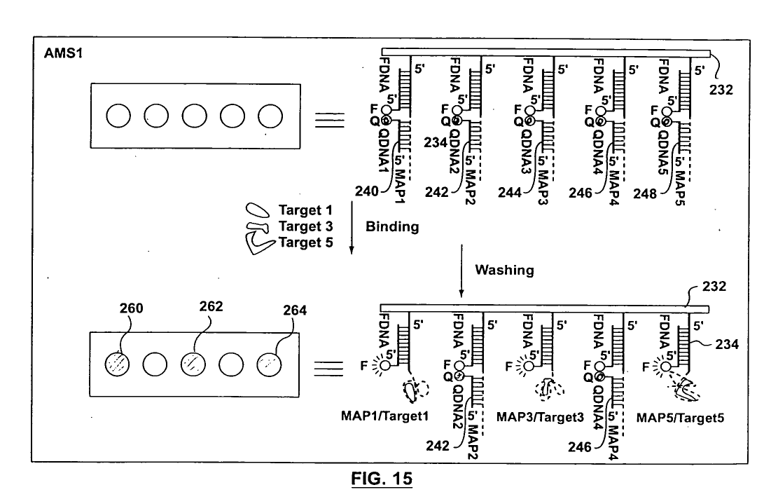

The present invention also provides for the construction of aptamer arrays for high throughput applications. FIG. 15 illustrates one exemplary configuration in which five signaling aptamers, converted according to AMS1, are deposited onto a glass surface 232. Since MAPs are standard oligonucleotides, they can be immobilized the same way as depositing synthetic DNA oligonucleotides to make DNA microarrays. The aptamer arrays can be hybridized with a solution that may contain the aptamer targets as well as added FDNA and QDNA. In the example illustrated in FIG. 15, the five aptamers all have a common FDNA-binding domain 234. Thus, a universal FDNA 236 can be used along with 5 different QDNAs 240, 242, 244, 246, and 248. The matching targets will be reported by the high intensity of fluorescence at particular spots. In the example given in FIG. 9, the targets for Aptamer 1, 3 and 5 are present in the solution, and they are identified by the increased fluorescence intensity at relevant spots 260, 262, 264 respectively. AMS1-2 and AMS4-8 are compatible with the strategy shown in FIG. 9 for the aptamer array construction because in all these schemes the quenchers will be released following the target binding and the fluorophore will be retained on the surface.

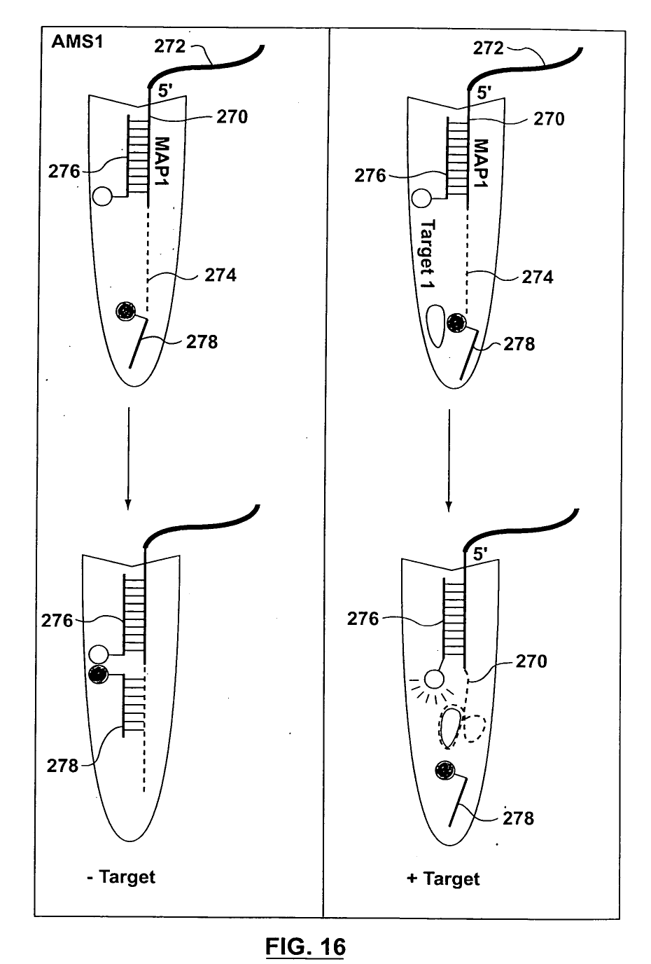

The signalling aptamers of the present invention can also be used to build optical sensors. FIG. 16 depicts an exemplary configuration using a fluorescent aptamer converted according to AMS1. The modified aptamer MAP1 270 is immobilized onto a glass tip 274 which is attached to an optical fiber 272 for fluorescence detection. The tip 274 is first dipped into a solution containing FDNA 276 and then is placed in a sample that may contain the target of interest (i.e., target 1). To the same sample, QDNA 278 is also added. As shown in panel A, in the absence of target, the QDNA 278 will anneal to MAP1 270, and as a result the fluorescence of the FDNA 276 is quenched and a weak signal will be recorded. When target 1 is present, as shown in Panel B, it will engage the aptamer sequence to form the tertiary structure, preventing QDNA 278 from being assembled onto MAP1 270. Since FDNA continues to fluoresce, a strong signal will be recorded. The setup is simple and the detection is instantaneous. Aptamer modification schemes, AMS1-2 and AMS4-8, are well-suited for the biosensor construction, as all these schemes involve the fluorescence generation by releasing the quenchers from the solid support. While fluoroscein and DABCYL were used in the construction of the signaling aptamer for ATP detection that is described in detail herein, it is clearly apparent that the methods of the present invention are not necessarily restricted to the use of these two chromophores as the fluorophore and the quencher and that other fluorophore-quencher pairs that can engage in efficient fluorescence quenching may also be used to make signalling aptamers.

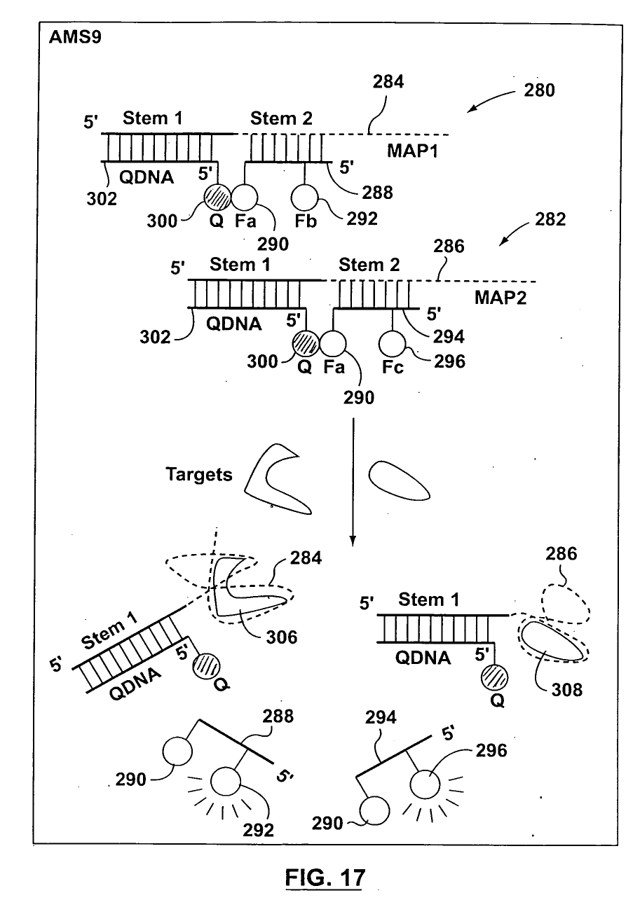

The modification schemes described herein are intended as exemplary methods of making fluorescent signalling aptamers based on a simple quenching-dequenching mechanism. It is clearly apparent that other, more complex fluorescence energy transfer strategies may also be used to generate signalling aptamer complexes based on structure switching. For example, FIG. 17 illustrates two wavelength-shifting signalling aptamers 280, 282 created using a modified version of AMS9. In the example provided, the first signalling aptamer complex 280 comprises a modified aptamer sequence termed MAP1 284 and the second signalling aptamer complex 282 comprises a modified aptamer sequence termed MAP2 286. The FDNA is doubly labeled with two flourophores (i.e. the FDNA 288 of the first signalling aptamer complex is labeled with Fa 290 and Fb 292 and the FDNA 294 of the second signalling aptamer complex is labeled with Fa 290 and Fc296. In the duplex structure state, the energy absorbed by the first fluorophore Fa 290 is not transferred to the second fluorophore (Fb 292 or Fc 296) but absorbed by the quencher 300 located within a shorter distance on QDNA 302 and therefore no fluorescence can be detected from the second fluorophores. In the presence of the cognate targets, MAP1 284 and MAP2 286 will form tertiary structures with target 1 306 and target 2 308, respectively. As a result of the target binding, the FDNAs 288, 294 are released into the solution. Since the energy absorbed by the first flurophore (Fa) 290 can now be transferred to the second fluorophore, Fb 292 or Fc 296 the characteristic fluorescence associated with the second fluorophores will be detected. Wavelength-shift signalling aptamer complexes with a common first-fluorophore and different second-fluorophores can be used to detect multiple targets in the same sample. Although several second-fluorophores are used, the sample only needs to be excited at a single wavelength characteristic of the common first-fluorophore without the need to excite all of the second-fluorophores. It is clearly apparent that various combinations of fluorophores and aptamer can be used.

The present invention is directed to signalling aptamer complexes in which the transition from a duplex structural state to a tertiary structure upon target binding can be detected by a change in a reporter signal. While the description has focussed on fluorescent reporters, it is clearly apparent that other types of reporter molecules could also be used. For example, a radioactively labelled DNA, “RDNA”, could be designed to be complementary to a segment of the aptamer sequence. In the presence of the cognate target, the RDNA would dissociate from the aptamer sequence and, upon washing, a decrease in radioactivity would be seen. This is merely an example. Various other reporter molecules could also be used to detect a switch in structure from duplex structure to tertiary structure.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

Methods of synthetic chemistry, protein and peptide chemistry and molecular biology, referred to but not explicitly described in this disclosure and examples are reported in the scientific literature and are well known to those skilled in the art.

### Example 1

### Oligonucleotides

Normal and modified oligonucleotides were all prepared by automated DNA synthesis using standard cyanoethylphosphoramidite chemistry (Keck Biotechnology Resource Laboratory, Yale University; Central Facility, McMaster University). Two kinds of modified oligonucleotides were prepared 5 that contained fluorescein and 4-(4-dimethylaminophenylazo)benzoic acid (DABCYL), respectively. Fluorescein and DABCYL were placed on the 5′ and 3′ ends of relevant oligonucleotides. 5′-fluorescein and 3′-DABCYL DNAs were synthesized by automated DNA synthesis with the use of 5′-fluorescein phosphoramidite and 3′-DABCYL-derivatized controlled pore glass (CPG) (Glen Research, Sterling, Va.). Unmodified DNA oligonucleotides were purified by 10% preparative denaturing (8 M urea) polyacrylamide gel electrophoresis (PAGE), followed by elution and ethanol precipitation. 5′-fluorescein or 3′-DABCYL modified oligonucleotides were purified by reverse phase high-pressure liquid chromatography (RP-HPLC). HPLC separation was performed on a Beckman-Coulter HPLC System Gold with 168 Diode Array detector. HPLC column was Agilent Zorbax ODS C18 Column, 4.5 mm×250 mm, 5-micron. Two buffer systems were used with buffer A being 0.1 M triethylammonium acetate (TEAR, pH 6.5) and buffer B being 100% acetonitrile. The best separation results can be achieved by a non-linear elution gradient (10% B for 10 min, 10% B to 40% B in 65 min) at a flow rate of 1 ml/min. The main peak was found to have very strong absorption at both 260 nm and 491 nm. The DNA within ⅔ peak-width was collected and dried under vacuum. Purified oligonucleotides were dissolved in water and their concentrations were determined spectroscopically. All chemical reagents were purchased from Sigma.

### Example 2

### Fluorescence Measurements

The following concentrations were used for various oligonucleotides (if not otherwise specified): 40 nM for fluorophores (FDNAs), 80 nM for aptamers (MAPs), 120 nM for the quenchers (QDNAs). All measurements were made in 1500 NI solutions containing 500 mM NaCl, 3.5 mM MgCl2 and 10 mM Tris.HCl (pH 8.3). The fluorescence measurement was undertaken on a Cary Eclipse Fluorescence Spectrophotometer (Varian) and with excitation at 490 nm and emission at 520 nm. To obtain the thermal denaturation profile of a particular reaction mixture, the DNA solution was heated to 90° C. for 5 min, and the temperature was then decreased from 90° C. to 20° C. at a rate of 1° C./min. A reading was made automatically for every 0.5° C. decrease.

### Example 3

### Standardized Solutions

A general three-step procedure for measuring the fluorescence intensity of samples was developed. The procedure comprises the following steps:
(1) Two 3× stock solutions were made and stored at −20° C., one of which (stock solution A) contained FDNA at 120 nM and MAP at 240 nM and the other (stock solution B) contained QDNA at 360 nM. The stock solutions also contained relevant metal ions and a buffer agent at desired concentration.
(2) The sample to be measured for ATP concentration was made to contain the same metal ions and the buffer agent at the same concentrations as used for the above two stock solutions.
(3) Stock solutions A and B were combined with the sample of interest at a ratio of 1:1:1. The resulting mixture was first incubated at 37° C. for 5 minutes and then let to stand at 22° C. for 10 minutes before its fluorescence was measured. The data obtained by the above procedure is highly reproducible with variation typically at below 10%.

### Example 4

### Construction of Test Signalling Aptamer Construct

In order to test the system, a 15-nt oligonucleotide modified with 5′ fluorescein (FDNA1) was used as the FDNA. A 15-nt oligonucleotide (QDNA1) having a quencher moiety at the 3′ end and a template DNA (template 1) were also prepared. As shown in FIG. 2a, the template, FDNA1 can form a linear duplex structure with the FB domain of the template and QDNA1 forms a duplex with the QB domain. The resulting two helical segments, stem 1 and stem 2, are separated by a single unpaired nucleotide (T). FDNA1 is GC rich in that 12 out of its 15 nucleotides are GCs. This provides for a very stable stem 1. The formation of the two helical structure elements brings the fluorophore and the quencher into close proximity and the fluorescence of FDNA1 is quenched when the three DNA oligonucleotides are mixed in solution. FIG. 2B illustrates the change in fluorescence intensity as a function of temperature. It is apparent that the properly annealed FDNA 1 and QDNA1 form a duplex assembly with fairly steady low fluorescence within the temperature range of 20° C. to 50° C. The data indicates that FDNA1 can form a duplex structure that is stable in the temperature range used for aptamer binding. As the temperature is increased the QDNA/QB domain duplex dissociates and an increase in fluorescence is seen as the quencher moves away from the fluorophore.

### Example 5

### Design of a Signalling Aptamer Complex

Once FDNA1 was established as a suitable FDNA, a modified DNA aptamer (MAP1) was synthesised that includes a tagging (FB) domain capable of hybridizing with FDNA1. An ATP-binding DNA aptamer was used as a model system. This 27-nt DNA aptamer was previously created using an in vitro selection approach (Huizenga & Szostak, 1995). This aptamer forms a tertiary complex with two ATP molecules. As shown in FIG. 3, a 15-nt GC-rich sequence was tagged onto the 5′-end of the aptamer for FDNA1 binding. A single nucleotide, T16 of MAP, was introduced to separate the FDNA1 binding domain (FB domain) and the aptamer domain (underlined) in order to minimize the potential steric interference between the two domains in the folded tertiary structure. Several 3′-DABCYL-modified oligonucleotides (QDNA1a to QDNA1d) were tested as quenchers [DABCYL: (4-(4-dimethylaminophenylazo)benzoic acid]. The ability of the varous QDNAs to form a stem-2 with MAP1 was assessed and the thermal denaturation profiles are shown in FIG. 3B. QDNA1b and QDNA1d were the two most effective quenchers in the group and had apparently equal quenching efficiency. This is likely because the 12-bp stem-2 formed by both QDNAs has the same base composition. The two 11-nt QDNAs, however, exhibited different quenching efficiencies with QDNA1c being more effective than QDNA1a. This may be due to the increased GC content of QDNA1c as it contains 8 GCs as compared to 7 GCs in QDNA1a. QDNA1c was chosen to test the ATP induced structure switching because QDNA1c forms a stem-2 that is almost as stable as those formed by the two 12-nt QDNAs at low temperatures (20° C.-30° C.). However, because QDNA1c has a less stable stem-2 (whose melting point is ˜3° C. lower than those by the two 12-nt QDNAS), it will dissociates more easily from MAP1 in the presence of ATP and provide a more sensitive reporter.

### Example 6

### Signalling Efficacy of a Model Aptamer Complex

The FDNA1-QDNA1c-MAP1 tripartite system is referred to as ATP Reporter A and is shown in FIG. 4A. A series of temperature-changing fluorescence assays were conducted to demonstrate the structure switching process. The results are shown in FIG. 4B. In each experiment, the pre-annealed ATP Reporter A was incubated at 15° C. for 10 minutes, followed by a rapid temperature increase (within 1 minute) from 15° C. to a designated temperature (37, 40, 45 or 55° C.), followed by a 50-minute incubation at each elevated temperature. Finally, the solution was rapidly cooled (within 1 minute) to 22° C. and incubated at this temperature for 30 minutes. In the absence of ATP as shown by the open symbols, the reporter had a low and stable fluorescence intensity at 15° C. When the temperature was raised from 15° C. to 37, 40, 45, or 55° C., the intensity of the solution increased in a manner that was indicative of heat denaturation of the DNA duplex assembly. A higher incubation temperature resulted in a higher fluorescence intensity because less and less QDNA1c remained as part of a duplex assembly. At each temperature, a stable fluorescence intensity value was re-established after a few minutes, indicating that the equilibrium between the amount of free QDNA1c and the amount of the QDNA1c bound in the DNA duplex assembly was reached. When the solution temperature was lowered to 22° C., the fluorescence intensity dropped owing to the re-association of some free QDNA1c molecules into the DNA duplex structure. The introduction of 1 mM ATP into the DNA mixture (filled data points) did not cause a rapid increase in fluorescence intensity at 15° C. and 22° C. However, when the temperature was raised from 15° C. to 37, 40, 45, or 55° C., rapid intensity increases were observed. This indicates that, as the aptamer structure switches to a tertiary structure upon binding to ATP, QDNA1c is dissociated from the complex. Referring now to FIG. 4C, t_{1/2} (the time required for the DNA solution to reach the half maximal fluorescence intensity after the addition of 1 mM ATP at a designated temperature) was determined to provide a quantitative measurement of the temperature dependence of the ATP-promoted intensity increase. The t_{1/2} at 22° C. was very large at 830 minutes; at 37° C., t_{1/2} was shortened to 6.8 minutes; when the temperature rose to 45° C., the half maximal intensity was reached in about 2 minutes. At temperature points other than 55° C., the presence of ATP caused a marked difference in the increase of fluorescence intensity. The contrast between the intensity changes of the ATP-containing and ATP-lacking solutions was even sharper when the temperature was lowered from each of the higher temperature points to 22° C. While the ATP-lacking solutions experienced a very significant decrease in fluorescence intensity, all the ATP-containing samples (including the one treated at 55° C.) registered a noticeable intensity gain. This is indicative of the stability of the target binding. The above observations are consistent with the structure switching mechanism shown in FIG. 1. Rapid structure switching did not occur at low temperatures (such as 15° C.) because most of the MAP1 molecules existed in the duplex form where the ATP binding site was partially occupied by QDNA1c. A rapid structural transition occurs at elevated temperatures because more QDNA1c molecules are forced to dissociate from the duplex assembly, and as a result, more free MAP1 molecules had their ATP-binding site freed for ATP binding. When the solution was cooled, QDNA1c molecules naturally re-annealed back onto the aptamer sequence in the absence of ATP. However, in the presence of ATP, the formation of the ATP-aptamer complex in the ATP-containing solution prevented the re-annealing of QDNA1c.

The ATP-aptamer binding is very stable despite the presence of QDNA1c. This is evident from the observation that the fluorescence intensity stayed unchanged upon continuous incubation at 22° C. from 62-90 minutes, as shown in FIG. 4B. The fluorescence intensity of each solution was measured after longer incubation times (up to 100 hours) and virtually no reduction in fluorescence intensity was found. These results indicate that ATP Reporter A is a highly effective signalling aptamer complex for the detections of a structural switch in the presence of ATP.

### Example 7

### Target Specificity

ATP Reporter A also demonstrates excellent sensing specificity as shown in FIG. 5. ATP Report A (FDNA1-QDNA1-MAP1) was incubated with 1 mM UTP, CTP, GTP, ATP and dATP. While 1 mM ATP resulted in ˜90% of the maximum fluorescence signaling capability as compared to the solution where the QDNA1c was omitted CTP, UTP or GTP at 1 mM were not able to induce significant intensity increases. The original ATP aptamer is known to bind dATP as well, and it was found that the signalling aptamer complex (ATP Report A) was able to bind to dATP. Furthermore, as shown in FIG. 6, double mutations within the ATP binding site of MAP1 (mutant M1 and mutant M2) abolished the ATP-binding capability. All of these observations are consistent with the specific ligand-dependent structural transition mechanism depicted in FIG. 1.

### Example 8

### Additional Exemplary Signalling Aptamers

The basic concept of the present invention can be easily expanded to include a variety of modification choices. FIG. 7A illustrates three more signalling aptamer complex duplex configurations. ATP Reporters B and C are bipartite systems involving the use of a fluorescein-dT (T1 and T15, respectively) as the fluorophore and a separate QDNA as the quencher. ATP Reporter D is another tripartite system where FDNA and QDNA were designed to bind two adjacent stretches of the unmodified DNA aptamer. The relevant QDNA and FDNA molecules were chosen for each configuration following the examination of thermal denaturation profiles of several constructs for each system (data not shown).

All four ATP reporters were tested for their signalling capability and specificity. The results are shown in FIG. 7B. Each of signalling aptamer complexes (ATP Reporters A-D) specifically detected the presence of ATP without false signaling for GTP (as well as CTP and UTP, data not shown). The results clearly illustrate that the aptamer modification system of the present invention can be used to design optimal signalling complexes for different aptamers.

### Example 9

### Insertion of Additional Nucleotides to Aptamer Sequence

To determine whether a reduction in the number of blocked nucleotides in the aptamer sequence from 11 to a smaller number (such as 6 or 7), would provide a reporter that works well at lower temperatures, additional nucleotides were introduced between the aptamer sequence and the FDNA-binding motif. As shown in FIG. 8A, a new tripartite signalling aptamer complex was designed which is referred to as ATP Reporter E. An arbitrary 5-nt sequence, CACGT, was inserted between the FDNA1-binding domain and the aptamer sequence (underlined). A 12-nt QDNA5 was used as the new quencher. QDNA5 forms base pairs with the five inserted nucleotides and the first seven nucleotides in the aptamer sequence making a bridging duplex. Referring now to FIG. 8B, ATP Reporter E was tested for real-time signalling at 15, 20, 25, and 37° C. The signalling complex was incubated at a designated temperature in the absence of ATP for 10 minutes, followed by the addition of 1 mM ATP and further incubation for 30 more minutes. ATP Reporter E was found to switch very quickly at all tested temperatures including 15° C. (the t_{1/2} for ATP Reporter E at all these temperatures was all less than 1 min). These data indicate that ATP Reporter E has a highly effective low-temperature real-time sensing capability. ATP Reporter E also provides a good signal to noise ratio. The signalling magnitude S/B,) is defined as the fluorescence intensity in the presence of ATP over that in the absence of any target. The S/B values were found to be 14.1, 13.0, 10.4, and 7.1 at 15, 20, 25 and 37° C., respectively, upon the addition of 1 mM ATP.

To determine whether the signal provided by ATP Reporter E is concentration sensitive, the fluorescence intensity achieved at 20° C. in the presence of different concentrations of ATP was measured over time. The real-time response is shown in FIG. 8C. These results indicated that ATP Reporter E is a highly efficient signalling aptamer complex and that the general modification scheme is feasible.

### Example 10

### Specificity and Dose Response of ATP Reporter E

The effect of ATP concentration on ATP Reporter E signalling was determined and the results are shown in FIG. 9A. The signal increased linearly as the ATP concentration was raised between 0.01-1 mM. This ATP Reporter E was also assessed for target specificity and the results are shown in FIG. 9B. While the reporter registered a large signalling magnitude in the presence of 1 mM ATP, ADP, and adenosine, the addition of 1 mM CTP (data not shown), 1 mM UTP (data not shown) or GTP did not induce a change in the fluorescence signal. 1 mM dATP and 1 mM AMP induced a smaller but still substantial fluorescence intensity increase (10-fold vs. 13-fold for 1 mM ATP). The intensity reduction was not caused by the inaccuracy of target concentrations as the concentration of each target was carefully determined by the standard spectroscopic methods. The reduced signaling magnitude seen with 1 mM dATP and AMP is apparently due to a shift in the saturating target concentration since the maximum fluorescence enhancement was achieved when 3 mM dATP or 3 mM AMP was used. The affinity of ATP Reporter E for AMP (and dATP) appears to be noticeably lower than that for ATP, ADP and adenosine. The above target specificity pattern is in good agreement with that observed for the original aptamer.

### Example 11

### Construction of an Anti-Thrombin Signalling Aptamer Complex

To demonstrate the general applicability of the above design strategy, a new reporter was engineered (using a DNA aptamer previously isolated for thrombin binding). As shown in FIG. 10A, a modified aptamer sequence, MAP6 was prepared. MAP6 contains the same FDNA1-binding domain further supporting the option of using FDNA1 as a general source of fluorophore. Seven nucleotides were inserted between the FDNA-binding domain and the aptamer sequence (underlined). A 12-nt QDNA, termed QDNA6, was used as the quencher. The signalling capacity of the modified anti-thrombin aptamer complex in response to structure switching was demonstrated using temperature-variation experiments similar to the ones discussed for ATP Reporter A (data not shown). The real-time signalling ability of the thrombin reporter complex also was assessed and the data are shown in FIG. 10B. Rapid signal generation was observed upon the addition of thrombin at 30° C. (t_{1/2}=1.4 min) and 37° C. (t_{1/2}=1.2 min). The reporter also exhibited a fairly rapid change in signal at 25° C. (t_{1/2}=4.6 min). The t_{1/2} tended to lengthen when the detection temperature was decreased further. The thrombin aptamer has a guanine-quartet based tertiary structure that is known to be sensitive to both metal ion identities and metal ion concentrations. A previous study has shown that while K⁺ promotes the formation of a stable aptamer-thrombin complex, other metal ions such as Mg²⁺ and Ca²⁺ do not support the complex formation [22]. The initial assaying mixture contained 1 mM MgCl₂ and 5 mM KCl. To determine whether the concentrations of potassium and magnesium ions might affect the real-time reporting capability of the thrombin reporter complex, a series of real-time sensing measurements was performed under different concentrations of KCl and MgCl₂. The results are shown in FIG. 10D. While changing potassium concentration between 1-5 mM did not significantly affect the real-time sensing ability of the reporter (data not shown), lowering magnesium concentration enhanced the reporter's real-time detection capability at room temperature considerably. FIG. 10C illustrates the signalling intensity of the thrombin reporter had a linear response to thrombin concentration over the range of 10-1000 nM and the maximum fluorescence enhancement reached nearly 12-fold.

Referring to FIG. 11, the target reporting was found to be very specific as other proteins, including bovine serum albumin (BSA), and human factors Xa and IXa, were not be able to generate fluorescence signals that were significantly above background.

1. A signalling aptamer complex for the detection of a target, the aptamer complex comprising:
i) a first oligonucleotide having a target binding domain, and
ii) at least one additional oligonucleotide having a sequence complementary to a region of said first oligonucleotide, wherein in the absence of the target, complementary regions of said first oligonucleotide and said additional oligonucleotide form a duplex structure and wherein in the presence of the target, said duplex structure dissociates and a reporter signal is generated.

2. The signalling aptamer of claim 1 wherein a reporter moiety is associated with the additional oligonucleotide and is selected from the group consisting of a fluorophore, a quencher, a radioactive marker, an enzyme and a density particle.

3. The signalling aptamer complex according to claim 1, wherein said first oligonucleotide is labeled with a fluorophore and said additional oligonucleotide has a quencher moiety associated therewith.

4. The signalling aptamer complex of claim 1, wherein said first oligonucleotide has a quencher moiety and said additional oligonucleotide is labeled with a fluorophore.

5. The signaling aptamer complex of claim 1, wherein said first oligonucleotide comprises an FDNA binding domain capable of forming a duplex with a fluorophore modified oligonucleotide (FDNA).

6. The signalling aptamer complex of claim 1, wherein said first oligonucleotide comprises 3-10 nucleotides inserted adjacent to the target binding domain wherein said nucleotides participate in the duplex formed between said first oligonucleotide and said additional oligonucleotide.

7. The signalling aptamer complex of claim 1, wherein the first oligonucleotide comprises an ATP-binding domain or a thrombin-binding domain.

8. A signalling aptamer complex for detection of a target, said aptamer complex comprising:
i) a first oligonucleotide having a target binding domain and a tagging domain,
ii) a second oligonucleotide labeled with a fluorophore and having a sequence complementary to said tagging domain, and
iii) a third oligonucleotide modified with a quencher and having a sequence complementary to a region of said target binding domain, wherein in the absence of a target, a first duplex is formed between said second oligonucleotide and said tagging domain and a second duplex is formed between said third oligonucleotide and a segment of said target binding domain whereby said quencher and said fluorophore are sufficiently close to one another to quench a fluorescent signal.

9. A signalling aptamer complex for detection of a target, said aptamer complex comprising:
i) a first oligonucleotide having a target binding domain and a tagging domain,
ii) a second oligonucleotide modified with a quencher and having a sequence complementary to said tagging domain, and
iii) a third oligonucleotide labeled with a fluorophore and having a sequence complementary to a region of said target binding domain, wherein in the absence of a target, a first duplex is formed between said second oligonucleotide and said tagging domain and a second duplex is formed between said third oligonucleotide and a segment of said target binding domain whereby said quencher and said fluorophore are sufficiently close to one another to quench a fluorescent signal.

10. A signalling aptamer complex according to claim 8, wherein said first oligonucleotide includes additional nucleotides intermediate said target binding domain and said tagging domain and said third oligonucleotide is complementary to and forms said second duplex with said additional nucleotides and the adjacent portion of the target binding domain.

11. A signalling aptamer complex according to claim 10 wherein, in the presence of a target, said first oligonucleotide assumes a tertiary structure and said third oligonucleotide dissociates from said first oligonucleotide and a fluorescent signal is detectable.

12. A signalling aptamer complex comprising:
i) a first oligonucleotide having a target binding domain
ii) a second fluorphore-labeled oligonucleotide hybridized to a first segment of the target binding domain, and
iii) a third quencher-modified oligonucleotide hybridized to a second segment of said target binding domain adjacent to the first segment.

13. A signalling aptamer complex according to claim 12, wherein said flurophore labeled oligonucleotide comprises two fluorophores capable of exhibiting fluorescence energy transfer.

14. A method for modifying an aptamer into a signalling aptamer, said method comprising interacting a reporter oligonucleotide, having a nucleotide sequence complementary to a target binding segment of the aptamer, with the aptamer to form a duplex structure.

15. The method of claim 14 wherein said aptamer is labeled with a fluorophore and said reporter oligonucleotide is modified with a quencher.

16. The method of claim 14 comprising modifying said aptamer to include a tagging domain at one end, forming a duplex between said tagging domain and a complementary fluorophore labeled-oligonucleotide, wherein said reporter oligonucleotide is modified with a quencher.

17. A method for detecting the presence of a target, said method comprising:
i) providing a signalling aptamer complex, as defined in claim 1;
ii) interacting said complex with a target solution; and
iii) measuring a signal.

18. A modified aptamer comprising a target binding domain and an oligonucleotide binding domain fused at one end.

19. A modified aptamer according to claim 18 wherein the oligonucleotide binding domain hybridizes to a flurophore-modified oligonucleotide.

20. A kit for the conversion of an aptamer to a signalling aptamer complex, said kit comprising a fluorophore labeled FDNA and a quencher modified QDNA.

21. A signalling aptamer complex according to claim 9, wherein said first oligonucleotide includes additional nucleotides intermediate said target binding domain and said tagging domain and said third oligonucleotide is complementary to and forms said second duplex with said additional nucleotides and the adjacent portion of the target binding domain.

22. A signalling aptamer complex according to claim 21 wherein, in the presence of a target, said first oligonucleotide assumes a tertiary structure and said third oligonucleotide dissociates from said first oligonucleotide and a fluorescent signal is detectable.

23. A method for detecting the presence of a target, said method comprising:
i) providing a signalling aptamer complex, as defined in claim 8;
ii) interacting said complex with a target solution; and
iii) measuring a signal.
